(19) United States
(12) Patent Application Publication
(10) Pub. No.: US 2008/0167248 A1
(43) Pub. Date: Jul. 10, 2008

(54) Anti-Aging Effects Of Substance P

(75) Inventor: Mark L. Witten, Tucson, AZ (US)

Correspondence Address: BANNER & WITCOFF, LTD., 1100 13th STREET, N.W., SUITE 1200, WASHINGTON, DC, 20005-4051, US

(73) Assignee: IMMUNEREGEN BIOSCIENCES, INC., Scottsdale, AZ (US)

(21) Appl. No.: 11/587,595

(22) PCT Filed: Apr. 18, 2005
(86) PCT No.: PCT/US2005/013113

§ 371 Date: Jan. 4, 2008

Related U.S. Application Data
(60) Provisional application No. 60/565,021, filed on Apr. 26, 2004.

(51) Int. Cl. A61K 38/08; A61P 27/00; A61P 21/00; A61P 25/00

(52) U.S. Cl. 514/15

(57) ABSTRACT

Substance P or its analogs can be administered to humans to reverse or retard the course of various biological processes related to aging in adults. Reversal or retardation can be observed in hair color, muscle or joint pain, memory loss, sleep pattern, and visual accommodation.

This application claims the benefit of provisional application Ser. No. 60/565,021 filed Apr. 26, 2004, the disclosure of which is expressly incorporated herein.

### FIELD OF THE INVENTION

The invention relates to treatments for reducing the effects of aging.

### BACKGROUND OF THE INVENTION

The process of aging has many negative affects on humans, albeit better than the alternative. Most old people suffer a decline in intelligence, learning abilities, short-term memory and reaction time. Age-related structural changes in the brain include weight decrease, loss of neurons, shrinkage of neurons, and loss of synapses in the frontal lobes. Ageing and its implications: an on-line primer for health care professionals and carers, http://www.healthandage.com/html/res/primer/index.htm.

Aging can have profound effects on a person's hair and image. With age, melanin is reduced (grey hair) or lost (white hair) from hair follicles. The age at which hair greys or falls out is genetically determined. Both women and men lose hair with age. Hair can becomes more brittle and downy with age, turning from terminal to vellus hair, and it grows more slowly. Older women may experience a conversion from vellus hair to terminal hair on their chins and above their upper lip. Older men may notice an increase in ear, nostril and eyebrow hair. Ibid.

Aging also negatively affects the eye. Typically, the eye lens thickens and becomes less elastic. This reduces the ability to focus, especially on near objects (presbyopia). The diameter of the pupil decreases and the iris around it becomes more fibrous. The eye therefore reacts more slowly to changes in light and darkness. Older people need increased illumination for optimal vision relative to younger people. Ibid.

Aging also affects the muscles, joints, and bones. These may often cause pain and lead to mobility problems. Strenuous exertion may cause residual pain, i.e., the pain which is perceived after exercising. Ibid.

Rheumatoid arthritis is an inflammation of the joints. It may be very sudden and severe, causing chronic and/ or extreme pain. Ibid.

The quality of sleep among older people often changes. It typically becomes shorter, lighter and more fragmented. Snoring is common and may disturb sleep. Ibid.

These and other age-related characteristics are well known in the field of geriatrics. There is a need in the art for treatments which will alleviate, reduce, and or ameliorate characteristics of aging in the humans.

### BRIEF SUMMARY OF THE INVENTION

In a first embodiment the invention provides a method of slowing or reversing age-related changes. An effective amount of substance P or a bioactive analog selected from the group consisting of:[Met-OH11]-substance P, [Met-OMe11]-substance P, [N1e11]-substance P, [Pro9]-substance P, [Sar9]-substance P, [Tyr8]-substance P, [p-C1-Phe7,8]-substance P, and [Sar9,Met (02)11]-substance P is administered to an adult human. One or more age-related characteristics are thereby slowed or reversed.

A second embodiment of the invention is a method of decreasing the ratio of grey or white to fully pigmented hair on a scalp of a human. An effective amount of substance P or a bioactive analog selected from the group consisting of:[Met-OH11]-substance P, [Met-OMe11]-substance P, [N1e11]-substance P, [Pro9]-substance P, [Sar9]-substance P, [Tyr8]-substance P, [p-C1l-Phe7,8]-substance P, and [Sar9,Met (02)11]-substance P is administered to a human whose scalp hair is greying. The ratio of pigmented hair to grey hair or the amount of pigment in the hair on the human's scalp is thereby increased.

A third embodiment of the invention provides a method of improving the sleep pattern of a human. An effective amount of substance P or a bioactive analog selected from the group consisting of:[Met-OH11]-substance P, [Met-OMe11]-substance P, [N1e11]-substance P, [Pro9]-substance P, [Sar9]-substance P, [Tyr8]-substance P, [p-C1-Phe7,8]-substance P, and [Sar9,Met (02)11]-substance P is administered to a human with a disturbed sleep pattern. Interruptions of sleep of the human are thereby decreased.

A fourth embodiment of the invention is a method of reducing residual muscle pain following exercise. An effective amount of substance P or a bioactive analog selected from the group consisting of: [Met-OH11]-substance P, [Met-OMe11]-substance P, [N1e11]-substance P, [Pro9]-substance P, [Sar9]-substance P, [Tyr8]-substance P, [p-Cl-Phe7,8]-substance P, and [Sar9,Met (02)11]-substance P is administered to a human who experiences residual muscle pain following exercise. Residual muscle pain in the human following exercise is thereby reduced.

Another aspect of the invention is a method of ameliorating short-term memory loss. An effective amount of substance P or a bioactive analog selected from the group consisting of: [Met-OH11] -substance P, [Met-OMe11] -substance P, [N1e11] -substance P, [Pro9]-substance P, [Sar9-substance P, [Tyr8]-substance P, [p-Cl-Phe7,8]-substance P, and [Sar9,Met (02)11]-substance P is administered to a human with short term memory loss. The memory loss is thereby diminished.

Still another aspect of the invention is a method of improving a human's visual accommodation. An effective amount of substance P or a bioactive analog selected from the group consisting of: [Met-OH11]-substance P, [Met-OMe11]-substance P, [N1e11]-substance P, [Pro9]-substance P, [Sar9]-substance P, [Tyr8]-substance P, [p-Cl-Phe7,8]-substance P, and [Sar9,Met (02)11]-substance P is administered to a human with diminished visual accommodation. The human's visual accommodation is thereby improved.

Yet another aspect of the invention is a method of increasing a human's muscle strength. An effective amount of substance P or a bioactive analog selected from the group consisting of:[Met-OH11]-substance P, [Met-OMe11]-substance P, [N1e11]-substance P, [Pro9]-substance P, [Sar9]-substance P, [Tyr8]-substance P, [p-Cl-Phe7,8]-substance P, and [Sar9,Met (02)11]-substance P is administered to a human. Muscle strength of the human is thereby increased.

According to still another aspect of the invention a method is provided for reducing pain due to arthritis in a human. An effective amount of substance P or a bioactive analog selected from the group consisting of:[Met-OH11]-substance P, [Met-OMe11]-substance P, [N1e11]-substance P, [Pro9]-substance P, [Sar9]-substance P, [Tyr8]-substance P, [p-Cl-Phe7,8]-substance P, and [Sar9,Met (02)11]-substance P is administered to a human with arthritis. Pain due to arthritis is thereby reduced in the human.

These and other embodiments of the invention provide the art with tools for combating some of the effects of aging.

### DETAILED DESCRIPTION OF THE INVENTION

It is a discovery of the present inventor that Substance P and its bioactive analogs, such as Sar9, Met (02) 11-Substance P, is a beneficial treatment for various characteristics related to the aging process. Such symptoms include: reduced ratio of pigmented to non-pigmented hair, interrupted sleep patterns, residual muscle pain following exercise, short-term memory loss, and loss of visual accommodation. In addition, pain due to arthritis (often associated with aging) is also reduced.

Substance P (RPKPQQFFGLM-NH2; SEQ ID NO: 1) or a bioactive analog thereof such as Sar⁹, Met(02)¹¹-Substance P can be administered to treat ARDS, SARS, corona and corona-like respiratory virus infections. The bioactive analog can be selected from the group consisting of [Met-OH11]-substance P, [Met-OMe¹¹]-substance P, [Nle11]-substance P, [Pro9]-substance P, [Sar9]-substance P, [Tyr8]-substance P, Sar9, Met (02) 11-Substance P, and [p-Cl-Phe7,8]-substance P. Other compounds which function in the same way can be identified by their ability to compete with substance P for binding to its receptors (NK-1, NK-2, and NK-3) or for their ability to agonize the NK-1 receptor. Routine assays for such activities are known in the art and can be used.

While not wanting to be bound by an particular mechanism of action or theory, it is believed that Sar9, Met (02)11-substance P, substance P, and its bioactive analogs affect expression of the daf-2 and sir-2 genes, and subsequently insulin growth factor. These two genes are thought to be involved in the aging process.

The substance P or analog can be administered by any method known in the art, including via aerosol inhalation. Intravenous, topical, intratracheal, intrabronchial, intramuscular, sublingual, and oral administrations can also be used. Preferred dosages include 0.05 to 5 nanomolar substance P or analog for administration, preferably 0.1 to 2 nanomolar, and more preferably 0.5 to 1.5 nanomolar. For aerosol administration dosages include 0.05 to 5 micromolar substance P or analog, preferably 0.1 to 2 micromolar, and more preferably 0.5 to 1.5 micromolar.

Typical concentration ranges of substance P or its bioactive analog in the aerosol administered is between 0.001 and 10 μM. It can be advantageously administered as a liquid at a concentration between about 0.1 and 10 μM. Concentrations for topical administration are in the range of 1 μM to 50 μM . Amounts to be administered are typically between 1 μM and 10 μM.

Bioactive analogs, according to the invention are those which act as competitive inhibitors of SP by binding to the SP receptor (NK-1 receptor). The analogs may be agonists of the NK-1 receptor or other neurokinin recptors. Other derivatives as are known in the art and commercially available (e.g., from Sigma) can be used. In addition, substance P fragments and derivatized substance P fragments may also be used. Substitution, deletion, or insertion of one to eight amino acid residues, and preferably from one to three amino acid residues, will lead to analogs which can be routinely tested for biological activity. In addition, functional groups may be modified on SP while retaining the same amino acid backbone. Again, routine testing will determine which of such modifications do not adversely affect biological activity.

Suitable devices for administering the aerosol of the present invention include nebulizers as well as hand-held aerosol “puffer” devices. Suitable treatment regimens for treatment according to the present invention include daily or multiple daily treatment by aerosol. Other modes of treatment include periodic topical applications, continual transdermal infusion, intravenous injection, intramuscular, sublingual, subcutaneous injection, and oral administration. Suitable formulations of substance P for administration are any which are pharmaceutically acceptable and in which substance P retains its biological activity. Generally, such formulations are substance P dissolved in normal sterile saline. Other formulations for changing absorption and half-life characteristics can be used, including gels, liposomal formulations and slow-release formulations. Vehicles which are typically used to apply to the skin may be used for formulating substance P and its analogs.

Age related characteristics or features can be reduced by a statistically significant amount. The change in measurable characteristics or features can be at least 10%, 15%, 20%, 25%, 30%, 35%, 40%, or 50%. Even greater decreases are preferred.

### EXAMPLES

[Sar9,Met (02)11 ]-substance P was formulated in a hair styling gel at a concentration of 0.001 mg/ml and approximately 2 ml were applied daily to my scalp. After a few months, I perceived that the overall color of my hair was becoming more like my original brown color, i.e., a higher percentage of the hairs are brown than before I started applying the substance P analog. I estimate my head of hair to be about 40% darker. This observation is consistent with results observed on radiation-exposed mice that had been given the substance P analog. The mice regrew hair that was lost upon radiation exposure and the new growth was darker than in the non-exposed area.

I have also observed a very large reduction in residual muscle pain which I typically experience after a very hard workout. I ran five miles with four 150M build-ups and threw a 6 Kg shot-put for the first time. This workout was followed by a session of weightlifting. Before I began treatment with the substance P analog I would take Advil™ at least twice on the day of the work out to prevent muscle pain. However, after taking the analog, I experienced no muscle pain after the hard workout, nor did I experience tightness or soreness in my right arm/shoulder from throwing the shot.

After taking the analog, I “felt” much faster in my 150M build-ups than previously.

In fact, when I timed myself in a 200M run, my time was significantly improved.

I have 6 hours sleep on a regular basis. Approximately a week ago, after taking the analog daily for 2 weeks, I was getting 5 hours sleep. The sleep seemed to be deeper with more vivid dreams and less “waking up.” I have now reverted back to six hours of sleep per night. Nonetheless, the vivid dreams and less waking up continue.

Since I began to apply the analog I seem to be having less “middle-aged” memory problems. I can remember people's names and telephone numbers more readily.

The adjustment time between my long and near vision is shorter and continues to diminish. After 97 days of administration, I could perceive little adjustment time between my long and near vision.

I claim:

1. A method of improving sleep pattern of a human, comprising:
administering an effective amount of substance P or a bioactive analog selected from the group consisting of: [Met-OH¹¹]-substance P, [Met-OMe¹¹-substance P, [Nle¹¹]-substance P, [Pro⁹]-substance P, [Sar⁹]-substance P, [Tyr⁸]-substance P, [p-Cl-Phe^{7,8}]-substance P, and [Sar⁹,Met (0₂)¹¹]-substance P to a human with a disturbed sleep pattern, whereby interruptions of sleep of the human are decreased.

2. A method of reducing residual muscle pain following exercise, comprising:
administering an effective amount of substance P or a bioactive analog selected from the group consisting of:[Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar⁹,Met (0₂)¹¹]-substance P to a human who experiences residual muscle pain following exercise, whereby the residual muscle pain is reduced following exercise.

3. A method of ameliorating short-term memory loss, comprising:
administering an effective amount of substance P or a bioactive analog selected from the group consisting of: [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar⁹,Met (0₂)¹¹]-substance P to a human with short term memory loss, whereby said memory loss is diminished.

4. A method of improving a human's visual accommodation, comprising:
administering an effective amount of substance P or a bioactive analog selected from the group consisting of: [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar⁹,Met (0₂)¹¹]-substance P to a human with diminished visual accommodation, whereby said accommodation is improved.

5. A method of increasing a human's muscle strength, comprising:
administering an effective amount of substance P or a bioactive analog selected from the group consisting of:[Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar⁹,Met (0₂)¹¹-substance P to a human, whereby muscle strength of the human is increased.

6. A method of reducing pain due to arthritis in a human, comprising:
administering an effective amount of substance P or a bioactive analog selected from the group consisting of: [Met-OH¹¹ ]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, and [Sar⁹,Met (0₂)¹¹]-substance P to a human with arthritis, whereby pain due to arthritis is reduced in the human.

7. The method of any of claims claim 1-6 wherein the substance P is administered topically.

8. The method of any of claims 1-6 wherein the substance P is administered via aerosol.

9. The method of any of claims 1-6 wherein the substance P is administered in a gel to skin.

10. The method of any of claims 1-6 wherein the substance P is administered sublingually.

11. The method of any of claims 1-6 wherein the substance P is administered intramuscularly.


SEQUENCE LISTING (1 sequence)

SEQ ID NO: 1 - protein, 11 aa, Homo sapiens
RPKPQQFFGLM